(19) United States
(12) Patent Application Publication
(10) Pub. No.: US 2021/0163997 A1
(43) Pub. Date: Jun. 3, 2021

(54) METHOD OF CREATING RECOMBINANT MICROORGANISM FOR MANUFACTURING FERMENTATION PRODUCT

(71) Applicants: Si-Yu Li, Taichung City (TW); Zhi-Xuan Shen, Taichung City (TW)

(72) Inventors: Si-Yu Li, Taichung City (TW); Zhi-Xuan Shen, Taichung City (TW)

(21) Appl. No.: 16/913,232

(22) Filed: Jun. 26, 2020

Related U.S. Application Data
(60) Provisional application No. 62/942,210, filed on Dec. 2, 2019.

(51) Int. Cl. C12P 7/06

(52) U.S. Cl. CPC C12P 7/065; C12R 1/19; C12Y 207/01019; C12Y 401/01039

(57) ABSTRACT

The present invention provides a method of creating a recombinant microorganism for manufacturing a fermentation product, comprising: providing a carbon fixing module; and providing a fermentation product producing module. Wherein the carbon fixing module comprises (a) providing the recombinant microorganism with knocking out an enzyme coding gene, and (b) providing a carbon dioxide to a ribulose-1,5-bisphosphate carboxylase/oxygenase and a phosphoribulokinase, and the fermentation product producing module comprises (c) a pyruvate only reacted with a pyruvate converting enzyme to produce the carbon dioxide. Furthermore, the present invention also provides a method of manufacturing the fermentation product, comprising utilizing aforementioned recombinant microorganism perform a fermentation process. The recombinant microorganism for manufacturing the fermentation product produced according to aforementioned method is also provided in the present invention.

### CROSS-REFERENCE TO RELATED APPLICATION

This application claims the benefit under 35 U.S.C. § 119(e) to the U.S. provisional patent application having the Ser. No. 62/942,210 filed on Dec. 2, 2019, the entirety of which is incorporated herein by reference.

### FIELD OF INVENTION

The present invention relates to a method of creating a recombinant microorganism. In particular, it relates to the method of creating the recombinant microorganism for manufacturing fermentation products.

### BACKGROUND OF THE INVENTION

Efforts to manipulate carbon fixation over the last decade have been extended beyond the scope of photoautotrophs by the genetic engineering of model heterotrophs, such as Escherichia coli and Saccharomyces cerevisiae. The use of engineered heterotrophs, which do not normally utilize CO₂ as a carbon source, complements the research conducted in autotrophs and offers an extended experimental toolbox to tackle the biotechnological challenge of the enhancement of the CO₂ fixation rate. In some previous researches, a mixotrophic E. coli was achieved by the heterologous expressions of Rubisco and Prk so that E. coli has been shown to be capable of the in situ recycling of CO₂ during the assimilation of pentose and glucose. An attempt has been made to quantify the performance of in situ CO₂ recycling by limiting the fermentation products to only C-2 (ethanol and acetate) and C-1 (formate and CO2). This allowed the use of a ratio of C-2/C-1 to monitor the in situ CO₂ recycling.

Bioethanol, an alternative biofuel, has emerged as the single largest biotechnology product, with about 100 billion liters produced worldwide in 2017. Bioethanol has been proposed to be the feedstock for the jet blendstock production. S. cerevisiae, Zymomonas mobilis, and E. coli were all used in homo-fermentative ethanol production. The first two of these produced ethanol naturally in high yield.

However, they only metabolize a few of the hexoses. This narrow range is a drawback because feedstock accounts for 10-80% of total production cost depending on the fluctuating and unstable feedstock price. In contrast, E. coli can assimilate a wide spectrum of the hexoses and pentoses present in low cost agricultural and forestry waste. However, E coli also produce mixed acids anaerobically, and the ethanol yield is usually low.

Genetic engineering (mutagenesis, specific gene knock-out, and metabolic manipulation) is a powerful approach to the elimination of these adverse traits and to turn these microorganisms into competitive ethanol producers with a theoretical ethanol yield of 85-95%.

The above information disclosed in this section is only for enhancement of understanding of the background of the described technology and therefore it may contain information that does not form the prior art that is already known to a person of ordinary skill in the art.

### SUMMARY OF THE INVENTION

Feedstock accounts for a major portion of bio-based chemical production. While tremendous progress has been made in cellulosic ethanol production over the last few decades, a direct conversion of CO₂ to biofuels is parallelly and vigorously under-investigated. In the present invention new, next generation, ethanol production is proposed. A recombinant strain of Escherichia coli has been constructed which is capable of mixotrophic ethanol production, where glucose and CO₂ are simultaneously converted to ethanol. To do this, the pflB gene (encoding pyruvate formate lyase) was knocked out of the E. coli MZLF (BL21(DE3) Δzwf ΔldhA Δfrd, in which the carbon flow to the oxidative pentose phosphate pathway, lactate, and succinate have been blocked) chromosome to obtain E. coli strain FB (MZLF ΔpflB). Then, the Pdc-mediated pathway was introduced to FB to obtain FB295 (FB containing pLOI295 (pdc and adhB)). Finally, the heterologous Rubisco-based engineered pathway consisting of rbcLS and prk (encoding ribulose-1,5-bisphosphate carboxylase/oxygenase (Rubisco) and phosphoribulokinase (Prk)) was introduced to FBL295 to construct FB295A. Practically homo-fermentative ethanol production can be achieved by FB295A in 60 hours where the ethanol yield, concentration, percentage/fermentation product, and CO₂ emission/ethanol production were 2.3±0.2 mol/mol, 256±19 mM, 100%, and 0.13±0.02 mol_{CO}_{₂}/mol_{EtOH}, respectively. By a sub-culture to the second generation, the overall fermentation time can be shortened within 30 h and therefore, the mixotrophic ethanol productivity was improved to 7.1±0.5 mmol·L⁻¹·h⁻¹ with the maintaining apparent ethanol yield of 2.4±0.1 mol/mol_{glucose}. The performance of FB295A reached 100% theoretical of the in situ CO₂ recycling.

To achieve aforementioned effect, the present invention provides a method of creating a recombinant microorganism for manufacturing a fermentation product, comprising: providing a carbon fixing module; and providing a fermentation product producing module. In particular, the carbon fixing module comprises (a) providing the recombinant microorganism with knocking out an enzyme coding gene, and (b) providing a carbon dioxide to a ribulose-1,5-bisphosphate carboxylase/oxygenase and a phosphoribulokinase. The fermentation product producing module comprises (c) a pyruvate only reacted with a pyruvate converting enzyme to produce the carbon dioxide.

In one embodiment of the present invention, the enzyme coding gene comprises zwf.

In one embodiment of the present invention, the step (c) further comprises knocking out frd, ldhA, and pflB from the recombinant microorganism.

In one embodiment of the present invention, the pyruvate converting enzyme is a pyruvate decarboxylase.

In one embodiment of the present invention, the recombinant microorganism is E. coli, Zymomonas mobilis, Cyanobacteria, Yeast, Bacillus or a combination thereof.

In one embodiment of the present invention, the fermentation product is a chemical derived from a reduction-oxidation reaction.

In one embodiment of the present invention, the chemical is an ethanol.

Moreover, the present invention also provides a method of manufacturing a fermentation product, comprising utilizing a recombinant microorganism according to aforementioned method to perform a fermentation process.

In addition, the present invention further provides a recombinant microorganism for manufacturing a fermentation product produced according to aforementioned method.

Many of the attendant features and advantages of the present invention will become better understood with reference to the following detailed description considered in connection with the accompanying drawings.

### BRIEF DESCRIPTION OF THE DRAWINGS

The detailed structure, operating principle and effects of the present invention will now be described in more details hereinafter with reference to the accompanying drawings that show various embodiments of the present invention as follows.

FIG. 1 illustrates metabolic pathways of mixotrophic ethanol production by E. coli FB295A (harboring the Pdc-mediated pathway).

FIG. 2 illustrates (a) The growth curve; (b) the glucose consumption; and (c) ethanol and acetate yields of strains FB, FBL, FB295, and FBL295. All strains were anaerobically grown in the MOPS minimal medium with 111 mM (20 g/L) glucose. Error bars represent standard deviation with a biological replicate of 3.

FIG. 3 illustrates (a) The growth curves; (b) glucose consumptions; and (c) the metabolite yields of E. coli strains when the Rubisco-based engineered pathway (designated as A in the last, if any) is implemented. All strains were anaerobically grown in the LB medium with approximate 111 mM (20 g/L) glucose for 60 h. Error bars represent standard deviation with biological replicates of 3 for FB295 and 6 for FB 295A.

FIG. 4(a) illustrates the growth curves of FB295 and FB295A with initial glucose concentrations of 69 and 108 mM; FIG. 4(b) and FIG. 4(c) illustrate time portion of OD₆₀₀ and glucose consumption of FB295A with initial glucose concentrations of 69 and 108 mM; and FIG. 4(d) represents apparent ethanol yields. A in the last of FB295A represents the Rubisco-based engineered pathway.

### DETAILED DESCRIPTION OF THE PREFERRED EMBODIMENTS

Reference will now be made in detail to the exemplary embodiments of the present invention, examples of which are illustrated in the accompanying drawings. Therefore, it is to be understood that the foregoing is illustrative of exemplary embodiments and is not to be construed as limited to the specific embodiments disclosed, and that modifications to the disclosed exemplary embodiments, as well as other exemplary embodiments, are intended to be included within the scope of the appended claims. These embodiments are provided so that this invention will be thorough and complete, and will fully convey the inventive concept to those skilled in the art.

For convenience, certain terms employed in the specification, examples and appended claims are collected here. Unless defined otherwise, all technical and scientific terms used herein have the same meaning as commonly understood by one of the ordinary skill in the art to which this invention belongs.

Various embodiments will now be described more fully with reference to the accompanying drawings, in which illustrative embodiments are shown. The inventive concept, however, may be embodied in various different forms, and should not be construed as being limited only to the illustrated embodiments. Rather, these embodiments are provided as examples, to convey the inventive concept to one skilled in the art. Accordingly, known processes, components, and techniques are not described with respect to some of the embodiments.

The singular forms “a”, “and”, and “the” are used herein to include plural referents unless the context clearly dictates otherwise.

The biological materials used in the present invention need not be deposited according to 37 CFR 1.802 since those biological materials are known and available to the public, or can be made, or isolated without undue experimentation.

With the awareness of sustainable future across communities and generations, metabolic engineering has been applied to developing recombinant strains for diverse feedstock usages. For example, bioethanol now can be produced from fermenting not only sugars and starchy but also lignocellulose and organic wastes. These newly developed recombinant strains have demonstrated the harmony between nature and industrial society. In the present invention, a new generation of ethanol production way is pursued. By combining gene sets from three bacterial species, i.e., cyanobacteria, Zymomonas mobilis, Escherichia coli, Yeast, Bacillus or a combination thereof, a recombinant E. coli can now convert sugars and CO₂ in an efficient way. Since the growth of the recombinant E. coli depends on both sugar and CO₂, the produced ethanol is called mixotrophic ethanol. Lastly, the mixotrophic behavior is a universal platform that can be employed for other bio-based chemical productions. Theoretically, the fermentation products can be any electron receiving chemicals.

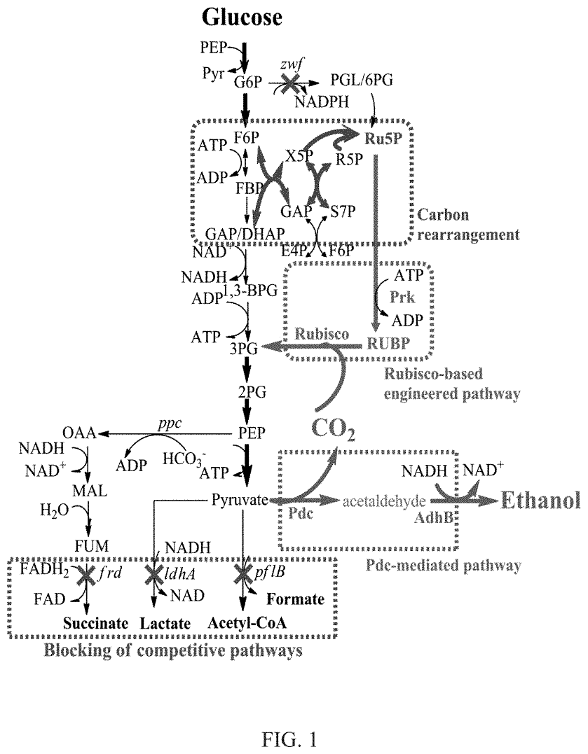

In the present invention, in order to achieve mixotrophic ethanol production, the E. coli strain FB295A was constructed (FIG. 1). Firstly, the gene pflB (encoding pyruvate formate lyase) was knocked out of the Escherichia coli chromosome MZLF to obtain E. coli strain FB (MZLF ΔpflB). Secondly, the Pdc-mediated pathway (pyruvate decarboxylase is used to perform it) was introduced to FB to obtain strains FB295 (FB containing pdc and adhB). Finally, the heterologous Rubisco-based engineered pathway consisting of rbcLS and prk (encoding ribulose-1,5-bisphosphate carboxylase/oxygenase (Rubisco) and phosphoribulokinase (Prk)) was introduced to FB295 to construct FB295A. The performance of FB295A for homo-fermentative and mixotrophic ethanol production was investigated and discussed.

The following descriptions are provided to elucidate a method of creating a recombinant microorganism for manufacturing a fermentation product and to aid it of skilled in the art in practicing this invention. These embodiments are merely exemplary embodiments and in no way to be considered to limit the scope of the invention in any manner.

### Material and Method

### Bacterial Strains and Plasmids

All the bacterial strains and plasmids used in the present invention, and the sources are listed in Table 1 as follows.

TABLE 1
List of bacterial strains and plasmids

| E. coli | | |
|---|---|---|
| strains | Descriptions | Available |
| MZLF | E. coli BL21(DE3) Δzwf, Δldh, | Yang et. al. |
| | Δfrd, impaired activities of oxidative | |
| | pentose phosphate pathway, lactate | |
| | production, succinate productions. | |
| FB | MZLF ΔpflB, impaired | The present |
| | activity of anaerobic conversion | invention |
| | of pyruvate to acetyl-CoA | |
| FBL | FB ΔpdhR::FRT-P_{pflB}, Integration | The present |
| | of P_{pflB} promoter in the chromosome | invention |
| | for the anaerobic expression of | |
| | pyruvate dehydrogenase complex. | |
| FB295 | FB harboring pLOI295, FB | The present |
| | strain containing the heterologous | invention |
| | ethanol producing pathway. | |
| FBL295 | FBL harboring pLOI295, FB | The present |
| | strain containing the heterologous | invention |
| | ethanol producing pathway. | |
| FB295A | FB harboring P_{BAD}-his6-prkA-pACYC184, | The present |
| | rbcLS-pET30a+, and pLOI295, | |
| | FB strain containing invention the Rubisco- | |
| | based pathway and the heterologous | |
| | ethanol producing pathway. | |
| Plasmids | | |
| P_{BAD}-his6- | Recombinant plasmid carries prkA | Prof. Ichiro |
| prkA- | gene (derived from Synechococcus | Matsumura |
| pACYC184 | PCC7942) for the overexpression of | |
| | phosphoribulokinase (Prk) under | |
| | the control of P_{BAD} promoter | |
| rbcLS- | Recombinant plasmid carries rbcLS | Prof. Ichiro |
| pET30a+ | gene (derived from Synechococcus PCC6301) | Matsumura |
| | for the overexpression of Rubisco | |
| | under the control of P_{T7} promoter | |
| pLOI295 | Recombinant plasmid carries pdc and | American |
| | adh gene (derived from Zymomonas | Type Culture |
| | mobilis) for the overexpression of pyruvate | Collection |
| | decarboxylase and alcohol dehydrogenase | (ATCC), |
| | II under the control of lac promoter | USA |
| pRED/ET | araC, bla, oriR101, repA101(Ts), γ, β, | Gene Bridges |
| | exo, RecA (red recombinase), temperature- | GmbH, |
| | conditional replicon | Germany |
| pKD13 | bla FRT-kan-FRT | Prof. |
| | | Yun-Peng |
| | | Chao |
| pCP20 | FLP+, λc1857+, λpR Pepts, bla, catF | Prof. |
| | | Yun-Peng |
| | | Chao |
| pTOL01 | Frt-kan-Frt region (1.3 kb) | The present |
| | amplified from pKD13 was inserted into | invention |
| | pCDFDuet-1 at NdeI and XhoI sites | |
| pTOL01P_{pflB} | pflB promoter region (0.30 kb) | The present |
| | amplified from BL21(DE3) genome | invention |
| | was inserted into pTOL01 at | |
| | XhoI and SalI sites | |

1. MZLF is obtained from Yang et. al. (C.-H. Yang, E.-J. Liu, Y.-L. Chen, F.-Y. Ou-Yang and S.-Y. Li, Microbial cell factories, 2016, 15, 133-133.)
2. Plasmids rbcLS-pET30a+ and P_{BAD}-his6-prkA-pACYC184 were obtained from Prof. Ichiro Matsumura of Department of Biochemistry at Emory University, USA.
3. Plasmids pKD13 and pCP20 were obtained from Prof. Yun-Peng Chao of Department of Chemical Engineering at Feng Chia University, Taiwan.

Cloning pTOL01P_{pflB}

The FRT-KAN-FRT gene fragment is amplified from pKD13 (using the primers FRTNde-F (SEQ ID NO: 1) and FRTXho-R (SEQ ID NO: 2)) and cloned between the NdeI-XhoI sites of the vector pCDFDuet-1 to yield pTOL01.

E. coli DH5α strain was used as a cloning host. The primer pair of pflB promoter-f (SEQ ID NO: 3)/pflB promoter-r (SEQ ID NO: 4) (please refer to Table 2) was used for the amplification of the pflB promoter DNA fragment from the E. coli BL21(DE3) chromosome. By adding the sequence of Pf1B ribosomal binding site to the primer, the resulting PCR product (P_{pf1B}) includes the transcriptional fusion of the Fnr box, pflBp6 promoter, and PflB ribosomal binding site. The P_{pflB} DNA fragment was then cloned into aforementioned pTOL01. E. coli DH5α and E. coli BL21(DE3) were obtained from Prof. Yun-Peng Chao of Department of Chemical Engineering at Feng Chia University, Taiwan. The primer sequences are listed in Table 2. Those primers are available from the service of Protech Technology Enterprise (Taiwan).

| | | SEQ ID |
|---|---|---|
| Primer | Sequence | NO: |
| FRTNde-F | GCATGCCATATGTGTAGGCTGGAGCTGCTTC | (1) |
| FRTXho-R | GACTCGCTCGAGGAATTAATTCCGGGGATCC | (2) |
| | G | |
| pflB | GGAGACTCGAGAACCATGCGAGTTACGGGCC | (3) |
| promoter-f | TATAA | |
| pflB | GGAGATTAATTAAGTAACACCTACCTTCTTG | (4) |
| promoter-r | TGCCTGTGCCAGTGGTTGCTGTGA | |
| pflB-HP1 | TGTCGAAGTACGCAGTAAATAAAAAATCCAC | (5) |
| | TTAAGAAGGTAGGTGTTACGTGTAGGCTGGA | |
| | GCTGCTT | |
| pflB-HP2 | GTGGAGCCTTTATTGTACGCTTTTTACTGTA | (6) |
| | CGATTTCAGTCAAATCTAAATTCCGGGGATC | |
| | CGTCGAC | |
| Gko-pflB | CTCCTTTCCTACGTAAAGTCTACATTTGTGC | (7) |
| promoter- | ATAGTTACAACTTTGTGTAGGCTGGAGCTGC | |
| HP1 | TTC | |
| Gko-pflB | GCGAGTTTCGATCGGATCCACGTCATTTGGG | (8) |
| promoter- | AAACGTTCTGACATGTAACACCTACCTTCTT | |
| HP2 | GTG | |

Construction of E. coli FB and FBL

The cloning of E. coli FB followed previously described procedures. A gene fragment was amplified from the template pKD13 using a pflB-HP1 (SEQ ID NO: 5)/pflB-HP2 (SEQ ID NO: 6) primer pair (please refer to Table 2). This gene fragment has a FRT-kan-FRT gene sequence at the center with which a 45-bp sequence of upstream of pflB coding region is fused at the 5′ end and a 45-bp sequence of downstream of pflB coding region is fused at the 3′ end. The amplified DNA was separated by electrophoresis (2.5 KV, 25 μF, and 200Ω) into E coli MZLF carrying pRed/ET and this was followed by screening for kanamycin resistant colonies. After verification of the integration of the kanamycin resistance marker in FB::FRT-kan-FRT by PCR, the marker was removed using FLP recombinase and the plasmid pCP20 to construct strain FB.

E. coli FBL produced by a gene knock-in process was developed in the present invention. A similar method to that used for FB, pTOL01P_{pflB} was used as the template for generating the linear fragment by PCR, the primer pair were Gko-pflBpromoter-HP1 (SEQ ID NO: 7)/Gko-pflBpromoter-HP2 (SEQ ID NO: 8) (please refer to Table 2). The amplified gene fragment had a FRT-kan-FRT-P_{plfB} sequence at the center with which a 45-bp sequence of upstream of pdhR coding region is fused at the 5′ end and a 45-bp sequence of aceE coding region is fused at the 3′ end. This linear DNA fragment was separated by electrophoresis into E. coli FB carrying pRed/ET for construction FB::FRT-kan-FRT-P_{pflB}. After verifying integration of the kanamycin resistance marker in FB::FRT-kan-FRT-P_{plfB} by PCR, it was removed by using FLP recombinase and the plasmid pCP20 to construct strain FBL.

In addition, MZLF is obtained from Yang et. al. (C.-H. Yang, E.-J. Liu, Y.-L. Chen, F.-Y. Ou-Yang and S.-Y. Li, Microbial cell factories, 2016, 15, 133-133.). FB 295 was produced by FB harboring pLOI295, and FBL was produced by FBL harboring pLOI295. Furthermore, FB295A was produced by FB harboring P_{BAD}-his6-prkA-pACYC184, rbcLS-pET30a+, and pLOI295. Those construction methods are conventional, and would not be repeated herein.

### Culture Media and Growth Conditions

Bacterial pre-cultures were grown in 5 ml LB broth at 37° C. with shaking at 200 rpm. Pre-cultures were used to inoculate 25 ml of LB (Becton, Dickinson and Company, USA) as a complex medium or Morpholino-propanesulfonic (MOPS, VWR Corporate, USA) as a defined medium in a 250 ml serum vial, to fix origin cell density at 0.05. MOPS contains: 0.4 mM MOPS; 0.04 mM Tricine; 0.1 mM FeSO₄.7H₂O; 95 mM NH₄Cl; 2.76 mM K₂SO₄; 0.005 mM CaCl₂.2H₂O; 5.25 mM MgCl₂; 500 mM NaCl; 0.029 mM (NH₄)₆Mo₇O₂₄.4H₂O; 0.004 mM H₃BO₃; 0.0003 mM CoCl₂; 0.0001 mM CuSO₄; 0.0008 mM MnCl₂; 0.0001 mM ZnSO₄; 1.32 mM K₂HPO₄. To ensure strictly anaerobic conditions, the headspace in the serum vials was filled with N₂ gas. Bacterial cells were cultured anaerobically at 37° C. with shaking at 200 rpm. Cell growth was monitored by measuring the optical density at 600 nm using a ThermoSpectonic GENESYSTM 10 Series spectrophotometer.

### Stoichiometry of CO₂ Conversion to Ethanol by the Rubisco-Based Engineered Pathway

Equation (a) shows the conventional stoichiometric reaction of the EMP pathway. Equation (b) represents a theoretical stoichiometric reaction describing the mixotrophic pyruvate production by carbon rearrangement and the rubisco-based engineered pathway (FIG. 1). Equation (c) represents a stoichiometric reaction of conventional ethanol production whereas Equation (d) is a stoichiometric reaction theoretically describing the mixotrophic ethanol production in E. coli.

Glucose+2 ADP +2 NAD⁺→2 pyruvate+2 ATP+2 NADH (a)

Glucose+1.2 CO₂→2.4 pyruvate (b)

Glucose+2 ADP→2 ethanol+2CO₂+2 ATP (c)

Glucose 30 2.4 NADH→2.4 ethanol+1.2 CO₂+2.4 NAD (d)

### Analysis of Metabolites

Characterization and quantification of glucose, formate, acetate, ethanol, lactate, succinate, and pyruvate were performed by using a Thermo Scientific™ Dionex™ Ultimate 3000 LC System. The separation of the mixture was achieved with HPLC column Aminex HPX-87H Column (300×7.8 mm, Bio-rad, USA) and measurement was done using refractive index (RI) or a UV detector. The mobile phase was 5 mM H₂SO₄. The temperature was maintained at 45° C. and the flow rate was 0.6 ml per minute. All samples were centrifuged for 5 minutes at 17,000×g to remove the cells and the supernatant was filtered using a 0.2 μm PVDF filter before the injection of a 10 μL sample by an autosampler.

The CO₂ gas concentration in the headspace of the cultures was measured using a Sentry ST303 diffusive infrared-based CO₂ analyzer. The method used for calculation of total CO₂ was done by a method previously described as follows.The gaseous CO₂ concentration in the headspace of the cultures was measured by a diffusive infrared-based CO₂ analyzer (Sentry ST303). The total CO₂ concentration was calculated based on the gaseous CO₂ concentration and the detailed calculation has been described as follows. The total amount of evolved CO₂ can be estimated by the following Equation:

CO_{2, total}(mole)=CO₂(1)+CO₂(g)+HCO₃⁻ (e)

Where CO_{2, total} (mole) is the total amount of evolved CO₂, CO₂ (e) is the total mole of CO₂ dissolved in the fermentation broth, CO₂ is the total mole of gaseous CO₂ in the headspace of the sealed fermentor, and HCO3⁻ is the total mole of bicarbonate ion in the fermentation broth. The total mole of gaseous CO₂ can be calculated by the volume of the headspace of the sealed fermentor (0.225 L) and the partial pressure of gaseous CO₂ measured by the diffusive infrared-based CO₂ analyzer (Sentry ST303).

### Results

An Intra-Cellular Redox Balance is Essential for Rescuing the Anaerobic Growth of E. coli FB in MOPS Minimal Medium

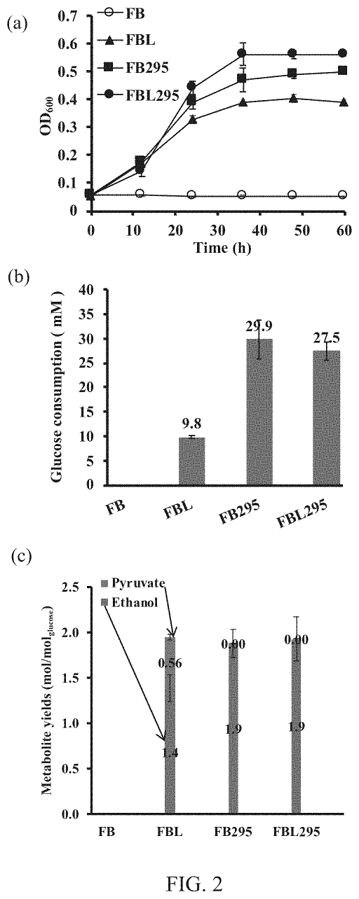

As shown in FIG. 2(a), E. coli FB (E. coli BL21(DE3) Δzwf, ΔldhA, Δfrd ΔpflB) was unable to anaerobically grow when glucose was the sole carbon source. This is consistent with some previous researches showing that the double mutation of ldhA and pflB causes a redox imbalance. To tackle the imbalance of redox potential, FBL was firstly constructed by introducing chromosomal-borne PDHc-mediated pathway in E. coli FB. In which the native promoter of PDHc was replaced with the endogenous pflB promoter (P_{pf}m, includes the transcriptional fusion of the Fnr box, pflBp6 promoter, and PflB ribosomal binding site), those techniques are conventional and not repeated herein. This allows the anaerobic expression of pyruvate dehydrogenase complex (PDHc), which otherwise is only expressed in the presence of oxygen. The PDHc in E. coli converts pyruvate to acetyl-CoA and CO₂, accompanied by the production of one NADH. However, the expression of PDHc, in the pdhR-aceEF-lpdA operon, is inhibited under anaerobic conditions. On the other hand, multiple genes and operons such as focA-pflB are readily expressed in E. coli in anaerobic conditions, under the global regulation of FNR. This characteristic makes P_{pflB} an ideal tool for driving gene transcription anaerobically. As shown in FIG. 2(a), strain FBL can anaerobically grow to OD₆₀₀ of 0.392±0.006 in 60 hours and was able to metabolize 9.8±0.0 mM glucose (FIG. 2(b)). Most of the glucose was converted to pyruvate and ethanol with a yield of 0.56±0.03 and 1.25±0.10 mol/mol_{glucose}, respectively (FIG. 2(c)).

When the Pdc-mediated pathway (pLOI295) was introduced to FB, the strain FB295 reached a OD₆₀₀ of 0.497±0.005 (FIG. 2(a)) with a glucose consumption of 29.9±0.2 mM at 60 h (FIG. 2(b)). FB295 produced ethanol with the yield of 1.8±0.2 mol/mol_{glucose}. No pyruvate, acetate, and formate was detected for the growth of FB295. FBL295 has a comparable fermentation profile to the one of FB295. An examination of FIG. 2 shows that the chromosomal-borne PDHc-mediated route is an alternative way for the rescue of impaired growth of FB in the absence of oxygen. Nevertheless, the accumulation of pyruvate (FIG. 2(c)) indicates the efficacy of the chromosomal-borne PDHc-mediated route can be further improved. Note that the growth of FBL, and FBL295 produced no formate.

Introduction of the Rubisco-Based Engineered Pathway Substantially Demonstrates Mixotrophic Ethanol Production in E. coli

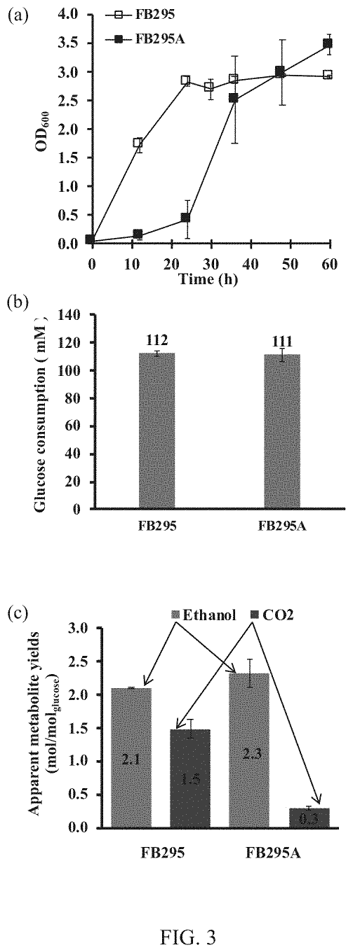

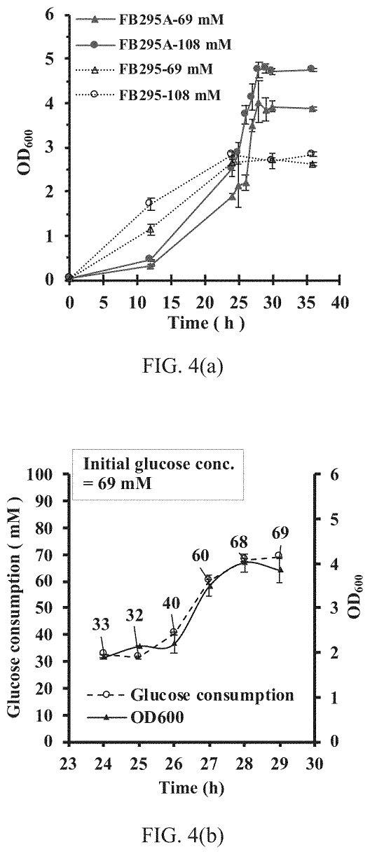

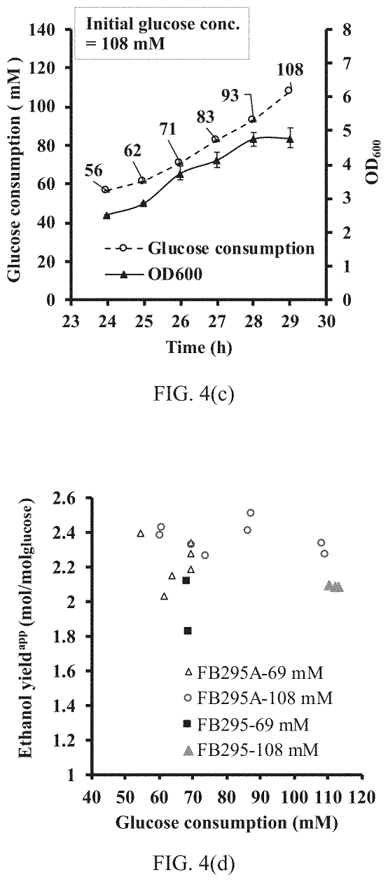

The growth of FB295 can be further improved when a complex medium was used. It can be seen in FIGS. 3(a) and 4(b) that FB295 entered stationary phase and consumed 112 mM glucose within 24 h. When NADH regeneration and ATP supply can be compensated by the use of LB medium, FB295 can efficiently convert glucose to ethanol without the co-factor regeneration. The apparent ethanol yield reached 2.09±0.01 mol/mol_{g}iucose, a 5% higher than the theoretical maximum.

When the Rubisco-based engineered pathway (containing Prk and Rubisco, designated as A) was introduced to strain FB295, a significant increase in the apparent ethanol yield (2.32±0.16 mol/mol_{glucose}) was observed in the resulting strain FB295A (p=0.043). On top of the apparent ethanol yield as a result of in situ CO₂ recycling (eq. d), ethanol was essentially the only fermentation product. The homo-fermentative ethanol production in FB295A was accompanied with an OD₆₀₀ of 4.03±0.08 (FIG. 3(a)) and a glucose consumption of 111±5 mM, consuming all the glucose provided in 60 hours (FIG. 3(b)). In addition, the CO₂ emission of FB295A was down to 0.3±0.03 mol/mol_{glucose}. Note that the final ethanol concentration of FB295A reached 256±19 mM.

### Sub-Culture of FB295A Demonstrates the Robust and Fast Mixotrophic Ethanol Production

This embodiment to verify whether the high yield of ethanol production can be maintained during the subculture of FB295A with a dilution rate of ca 2.5%. In addition to the initial glucose concentration of 108 mM (ca 20 g/L), 69 mM of initial glucose concentration was also tested to mimic the common glucose concentration found in the hydrolysate of lignocellulose. It can be seen in FIG. 4(a) that one-time sub-culture (2^{nd} generation) of FB295A reached to the stationary phase within 29 h, which was significantly improved compared to the 1^{st} generation (shown in FIG. 3(a)). FIGS. 4(b) and 4(c) showed a high positive correlation between OD₆₀₀ and glucose consumption regardless of the initial glucose consumption. Furthermore, FIG. 4(d) showed the apparent ethanol yield of FB295A can be maintained almost above 2.2 mol/mol_{glucose} throughout the glucose consumption from 50 to 108 mM. This indicates that the apparent ethanol yield of FB295A is independent of fermentation time and also indicates that mixotrophic ethanol is substantially a primary metabolite. The average glucose consumption rate of FB295A (108 mM initial glucose concentration) can be calculated to be 3.0±0.2 mmol·L⁻¹·h⁻¹ using the least square method with a zero intercept. Also calculated was the apparent ethanol yield of FB295A (108 mM initial glucose concentration) from FIG. 4(d) and was 2.4±0.1 mol/mol_{glucose}. Therefore, the mixotrophic ethanol productivity was 7.1±0.5 mmol·L⁻¹·h⁻¹ with the initial glucose concentration of 108 mM. Ethanol was the only fermentation product found in the liquid broth.

### Discussion

The two insertional inactivations of ldhA and pflB in E. coli FB result in a redox imbalance and a failure in the anaerobic fermentation of glucose (FIG. 2(a)). The introduction of the pyruvate oxidizing PDHc-mediated and Pdc-mediated pathways, successfully rescued the growth of FB (FIG. 2(a)). This rescue of FBL, FB295 and FB295A bacterial growth was accompanied by a significant amount of ethanol production (FIG. 2(c)). The impaired growth of FB can be attributed to the redox imbalance which can be fixed by ethanol production through the PDHc-mediated or Pdc-mediated route. The high growth and high glucose consumption of FB295 compared to that of FBL indicated that the ATP demand created by the Pdc-mediated pathway is an effective approach to stimulation of glucose consumption (Eq. c). This is because the Pdc-mediated pathway only produces ethanol rather than acetate so that the EMP pathway becomes the major route for ATP production. On the other hand, the PDHc-mediated pathway produces acetate with ATP as a byproduct. The ATP-producing nature of the PDHc-mediated pathway makes it an inefficient driving force for stimulating glucose consumption. Therefore, FB strain was used for the rest. Note that the growth behavior of FB (failure to grow anaerobically) shown in FIG. 2 exemplifies how intra-cellular energy balances bacterial growth.

The strain FB295 can be grown in the MOPS minimal medium (FIG. 2); however, strain FB295A can only be grown in the LB complex medium when the Rubisco-based engineered pathway was introduced in FB295 (data not shown). Glucose can be routed to either the native EMP pathway or the Rubisco-based engineered pathway (FIG. 1). Competition in carbon flow is essentially a dilemma for in situ CO₂ recycling because the EMP pathway is the major route for ATP generation. As described in Eq. d, the mixotrophic ethanol production requires NADH, which cannot be explicitly supplied from the MOPS minimal medium. Eq. d also indicates that there is no source of ATP production. Therefore, the non-growth of FB295A in the minimal medium suggested that FB295A had a strong in vivo Prk activity of the Rubisco-based engineered pathway but lacked a sufficient ATP and NADH supplies. In fact, the strong activity of Prk can serve a robust driving force to direct the glucose flow to the Rubisco-based engineered pathway from the conventional EMP pathway. This can be supported that the Rubisco-based engineered pathway becomes the major route in FB295A to efficiently ferment glucose to ethanol (eq. d) when the LB complex medium was used. The use of the complex medium made the supply of ATP and NADH implicitly feasible.

One potent characteristic of FB295A is that more than 100 mM of glucose had been consumed in LB medium within 60 hours while the bacteria were still in the exponential phase (FIG. 3(a)). This shows clearly that glucose and CO₂ are assimilated during growth. More strong evidence can be found in FIGS. 4(b)-4(d) where a high sampling frequency reveals that a positive correlation among OD₆₀₀, the glucose consumptions, and the high apparent ethanol yields. The other important characteristic of FB295A is that overall fermentation time can be significantly reduced by a sub-culture to the second generation while mixotrphic ethanol production is maintained (apparent ethanol yield of 2.4±0.1 mol/mol_{glucose}. By comparing FIGS. 3(a) (generation 1) and 4(a) (generation 2), the accelerated fermentation time is mainly because of the reduced lag phase. This indicates that the Rubisco-based engineered pathway for ethanol production (eq. d) is easily compatible to the current metabolic infrastructure of heterotrophic E. coli. The apparent ethanol yield of 2.4±0.1 mol/mol_{glucose} indicates that glucose is metabolized completely through the Rubisco-based engineered pathway (as depicted in Eq. d). As reviewed in the section of background of the invention that some extensive researches have been reported to reach 85-95% of theoretical maximum (the conventional ethanol fermentation as depicted in Eq. c), the present invention investigates a novel pathway for ethanol production (Eq. d) and reaches a 100% of theoretical maximum. While the chemistry of mixotrophic ethanol production is presented in the present invention, the use of less expensive and defined media in a bioreactor could be further investigated for an economic and competitiveness of mixotrophic ethanol production. Moreover, the mixotrophic ethanol productivity is expected to be improved through the proper use of the bioreactor.

It should be noted that the strong activity of the Rubisco-based engineered pathway in FB295A indicates the expression of Rubisco in E. coli is not a rate-limiting step for in situ CO₂ recycling. In fact, Rubisco is not only known for its low k_{cat} but also known for being inhibited by its own substrate ribulose-1,5-bisphosphate (RuBP). The carboxylation power of Rubisco should be based on the carbamylation of Rubisco. The RuBP inhibition occurs when RuBP binds to Rubisco and forms a conformation to prevent the carbamylation. In the present invention, the introduction of the Pdc-mediated pathway potentially provides one advantage for in situ CO₂ recycling, i.e., CO₂ supply. The vigorous CO₂ supply may overcome in vivo RuBP inhibition and strengthen the activity of the Rubisco-based engineered pathway. While enoyl-CoA carboxylases/reductases (Ecrs) was arguably the most competitive in terms of carboxylation capability, the present invention demonstrated the practical and potent role of Rubisco in the engineering perspective.

To sum up, a homo-fermentative ethanol production can be achieved by FB295A in 60 hours where the yield, concentration, and purity of the ethanol in the fermentation product, and CO₂ emission/ethanol production were 2.3±0.2 mol/mol, 256±19 mM, 100%, and 0.13±0.02 mol_{CO}_{₂}/mol_{EtOH}, respectively. By a sub-culture to the second generation, the overall fermentation time can be shortened within 30 h and therefore, the mixotrophic ethanol productivity was improved to 7.1±0.5 mmol·L⁻¹·h⁻¹ with the apparent ethanol yield of 2.4±0.1 mol/mol_{glucose}. The performance of FB295A reached 100% theoretical of the performance of in situ CO₂ recycling as depicted in Eq. d.

It will be understood that the above description of embodiments is given by way of example only and that various modifications may be made by those with ordinary skill in the art. The above specification, examples, and data provide a complete description of the present invention and use of exemplary embodiments of the invention. Although various embodiments of the invention have been described above with a certain degree of particularity, or with reference to one or more individual embodiments, those with ordinary skill in the art could make numerous alterations or modifications to the disclosed embodiments without departing from the spirit or scope of this invention.

1. A method of creating a recombinant microorganism for manufacturing a fermentation product, comprising:
providing a carbon fixing module; and
providing a fermentation product producing module,
wherein the carbon fixing module comprises (a) providing the recombinant microorganism with knocking out an enzyme coding gene, and (b) providing a carbon dioxide to a ribulose-1,5-bisphosphate carboxylase/oxygenase and a phosphoribulokinase,
wherein the fermentation product producing module comprises (c) a pyruvate only reacted with a pyruvate converting enzyme to produce the carbon dioxide.

2. The method of claim 1, wherein the enzyme coding gene comprises zwf.

3. The method of claim 1, wherein the step (c) further comprises knocking out frd, ldhA, and pflB from the recombinant microorganism.

4. The method of claim 1, wherein the pyruvate converting enzyme is a pyruvate decarboxylase.

5. The method of claim 1, wherein the recombinant microorganism is E. coli, Zymomonas mobilis, Cyanobacteria, Yeast, Bacillus or a combination thereof.

6. The method of claim 1, wherein the fermentation product is a chemical derived from a reduction-oxidation reaction.

7. The method of claim 6, wherein the chemical is an ethanol.

8. A method of manufacturing a fermentation product, comprising utilizing a recombinant microorganism according to claim 1 to perform a fermentation process.

9. The method of claim 8, wherein the fermentation product is a chemical derived from a reduction-oxidation reaction.

10. The method of claim 9, wherein the chemical is an ethanol.

11. A recombinant microorganism for manufacturing a fermentation product produced according to the method as defined in claim 1.


SEQUENCE LISTING (8 sequences)

SEQ ID NO: 1 - DNA, 31 bp, Artificial Sequence
  FRTNde-F
gcatgccatatgtgtaggctggagctgcttc

SEQ ID NO: 2 - DNA, 32 bp, Artificial Sequence
  FRTXho-R
gactcgctcgaggaattaattccggggatccg

SEQ ID NO: 3 - DNA, 36 bp, Artificial Sequence
  pflB promoter-f
ggagactcgagaaccatgcgagttacgggcctataa

SEQ ID NO: 4 - DNA, 55 bp, Artificial Sequence
  pflB promoter-r
ggagattaattaagtaacacctaccttcttgtgcctgtgccagtggttgctgtga

SEQ ID NO: 5 - DNA, 69 bp, Artificial Sequence
  pflB-HP1
tgtcgaagtacgcagtaaataaaaaatccacttaagaaggtaggtgttacgtgtaggctggagctgctt

SEQ ID NO: 6 - DNA, 69 bp, Artificial Sequence
  pflB-HP2
gtggagcctttattgtacgctttttactgtacgatttcagtcaaatctaaattccggggatccgtcgac

SEQ ID NO: 7 - DNA, 65 bp, Artificial Sequence
  Gko-pflBpromoter-HP1
ctcctttcctacgtaaagtctacatttgtgcatagttacaactttgtgtaggctggagctgcttc

SEQ ID NO: 8 - DNA, 65 bp, Artificial Sequence
  Gko-pflBpromoter-HP2
gcgagtttcgatcggatccacgtcatttgggaaacgttctgacatgtaacacctaccttcttgtg